(19) United States
(12) Patent Application Publication
(10) Pub. No.: US 2021/0322723 A1
(43) Pub. Date: Oct. 21, 2021

(54) SYSTEMS AND METHODS FOR TREATING PATENT FORAMEN OVALE

(71) Applicant: Subramaniam Chitoor Krishnan, Sacramento, CA (US)

(72) Inventor: Subramaniam Chitoor Krishnan, Sacramento, CA (US)

(21) Appl. No.: 17/251,115

(22) PCT Filed: Jul. 2, 2019
(86) PCT No.: PCT/US2019/040401

§ 371 Date: Dec. 10, 2020

Related U.S. Application Data
(60) Provisional application No. 62/693,509, filed on Jul. 3, 2018.

(51) Int. Cl. A61M 25/00; A61B 18/14; A61M 25/10

(52) U.S. Cl. CPC A61M 25/0084; A61B 2018/0038; A61M 25/1011; A61B 18/1477

(57) ABSTRACT

Systems, apparatuses, and methods for treating a patent foramen ovale (PFO) of a patient. A system may include a catheter configured to extend within at least a portion of the patients heart. One or more stabilizer balloons may be coupled to the catheter and configured to be positioned within a tunnel of the patent foramen ovale and inflated to apply a force to at least one of a septum primum or a septum secundum of the patients heart. An injection device may be configured to inject material into at least one of the septum primum or the septum secundum to cause swelling of the injected septum primum or the injected septum secundum in a direction towards the opposing septum primum or the opposing septum secundum.

### CROSS REFERENCE TO RELATED APPLICATIONS

This application claims the benefit of U.S. Provisional Application No. 62/693,509, filed Jul. 3, 2018, the entire contents of which are incorporated by reference herein.

### BACKGROUND

A patent foramen ovale (PFO) is a cardiac condition present in a large number of adult individuals. It comprises an opening or communication between the right and left atria of the heart that did not fully close naturally and appears as an oblique slit-like tunnel. The presence of such a condition may be harmless for most individuals, but in some, it may have serious consequences including the possibility of migraine headaches, decompression sickness, and stroke due to a blood clot passing through the PFO.

Systems have been developed to artificially close the PFO. A current clinical standard for PFO closure is the transvenous implantation of an occluding device. However, a medical implant left permanently within the patient's heart to close the PFO can be associated with problems. These include the possibility of the implant becoming dislodged or being improperly inserted during initial deployment. Short term and long term complications can include effects caused by the device itself and its potential interaction with the surrounding structures: the occluding device may trigger atrial fibrillation and thus may induce the need for long-term oral anticoagulation. In addition, the surface of the occluding device may serve as a source of clot formation; there is a possible risk of erosion into other cardiac structures such as the aorta or the roof of the left atrium. Furthermore, any future interventional catheter treatment which requires left atrial access has either to bypass the occluding device or directly puncture and perforate the device itself, which therefore complicates any left atrial intervention, e.g., atrial fibrillation ablation, appendage occluder implantation, or mitral valve clipping procedures.

There is accordingly a need for methods, and apparatuses and systems thereof; that can achieve a PFO closure without having to permanently leave behind an occluding device.

### SUMMARY

Systems, apparatuses, and methods disclosed herein may be directed to treating the presence of a PFO. The systems, apparatuses, and methods disclosed herein may comprise an improvement of the systems, apparatuses, and methods described in U.S. Publication No. 2008/0221566 to Krishnan, for “Method and Apparatus for Detecting and Achieving Closure of Patent Foramen Ovale.” The systems, apparatuses, and methods disclosed herein may utilize one or more balloons to apply a force to a portion of a patient's heart, in a similar manner as described in U.S. Publication No. 2016/0166242 to Krishnan for “Methods and Systems for Preventing Bleeding from the Left Atrial Appendage.”

The systems, apparatuses, and methods disclosed herein may utilize fusion of tunnel surfaces of the septum secundum and the septum primum to close the PFO, rather than installing an implant to close the PFO. The tunnel surfaces may be fused due to ablation of the tunnel surfaces that leads to injury, inflammation, and endothelial denudation of the tunnel surfaces. The ablated or raw surfaces may then come in contact with each other to fuse to each other, to treat and close the PFO.

Systems, apparatuses, and methods disclosed herein may be directed to causing the ablated or raw surfaces to come in contact with each other, to lead to fusion and treatment and closure of the PFO. Systems, apparatuses, and methods disclosed herein may include pushing the ablated or raw surfaces towards each other, to lead to fusion and treatment and closure of the PFO.

Systems, apparatuses, and methods disclosed herein may be directed to injecting one or more of the septum secundum or the septum primum with a material causing swelling of the injected septum secundum or injected septum primum. The injected septum secundum or septum primum may swell towards the opposing septum secundum or opposing septum primum and come in contact with the opposing septum secundum or opposing septum primum. Fusion and treatment and closure of the PFO may then occur. Systems, apparatuses, and methods disclosed herein may be directed to stabilizing the injected septum secundum or injected septum primum to provide a more accurate injection of the material into the septum secundum or the septum primum, which may include varying degrees of depth. The stabilization may immobilize and fix the septum secundum or septum primum in position.

A system is disclosed herein for treating a patent foramen ovale. The system may include a catheter configured to extend within at least a portion of the patient's heart. The system may include one or more stabilizer balloons coupled to the catheter and configured to be positioned within a tunnel of the patent foramen ovale and inflated to apply a force to at least one of a septum primum or a septum secundum of the patient's heart. The system may include an injection device configured to inject material into at least one of the septum primum or the septum secundum to cause swelling of the injected septum primum or the injected septum secundum in a direction towards an opposing septum primum or an opposing septum secundum. In one embodiment, the system may be utilized for treating a channel like structure in the body. The channel like structure may be similar to the patent foramen ovale.

An apparatus is disclosed herein for treating a patent foramen ovale of a patient. The apparatus may include a catheter configured to extend within at least a portion of the patient's heart. The apparatus may include one or more stabilizer balloons coupled to the catheter and configured to extend outward from the catheter to apply a force to at least one of a septum primum or a septum secundum of the patient's heart, the one or more stabilizer balloons having an opening. The apparatus may include an injection device configured to extend through the opening to inject material into at least one of the septum primum or the septum secundum to cause swelling of the injected septum primum or the injected septum secundum in a direction towards an opposing septum primum or an opposing septum secundum. In one embodiment, the apparatus may be utilized for treating a channel like structure in the body. The channel like structure may be similar to the patent foramen ovale.

A method is disclosed herein for treating a patent foramen ovale of a patient. The method may include stabilizing at least one of a septum primum or a septum secundum of the patient's heart. The method may include injecting at least one of the septum primum or the septum secundum with material to cause swelling of the injected septum primum or the injected septum secundum in a direction towards an opposing septum primum or an opposing septum secundum.

### BRIEF DESCRIPTION OF THE DRAWINGS

Other systems, methods, features, and advantages of the present disclosure will be apparent to one skilled in the art upon examination of the following figures and detailed description. Component parts shown in the drawings are not necessarily to scale and may be exaggerated to better illustrate features of the present disclosure.

FIG. 1 illustrates a modified coronal sectional view of a patient's heart with a patent foramen ovale.

FIG. 2 illustrates close up view of a portion of FIG. 1 showing the two components of the foramen ovale i.e. a septum primum and a septum secundum that are separate and not fused.

FIG. 3 illustrates a modified coronal sectional view of a patient's heart where an ablation catheter is placed against the opposing tunnel surfaces of the septum primum and secundum.

FIG. 4 illustrates a modified coronal sectional view of a patient's heart where ablation has occurred to the opposing tunnel surfaces of the septum primum and secundum.

FIG. 5 illustrates close up view of a portion of FIG. 4.

FIG. 6 illustrates a side cross sectional view of an apparatus according to an embodiment of the present disclosure.

FIG. 7 illustrates a side view of an apparatus according to an embodiment of the present disclosure.

FIG. 8 illustrates a modified coronal sectional view of a patient's heart with the apparatus shown in FIG. 7 extending therein.

FIG. 9 illustrates a side cross sectional view of an apparatus according to an embodiment of the present disclosure.

FIG. 10 illustrates a close up side view of the apparatus shown in FIG. 9 within a patient's heart.

FIG. 11 illustrates a side cross sectional view of an apparatus according to an embodiment of the present disclosure.

FIG. 12 illustrates a modified coronal sectional view of a patient's heart with the apparatus shown in FIG. 11 extending therein.

FIG. 13 illustrates a close up side view of the apparatus shown in FIG. 11 within a patient's heart.

FIG. 14 illustrates a top cross sectional view of a patent foramen ovale.

FIG. 15 illustrates a top cross sectional view of a patent foramen ovale with the apparatus shown in FIG. 11 positioned therein.

FIG. 16 illustrates a top cross sectional view of a patent foramen ovale with the apparatus shown in FIG. 11 positioned therein.

FIG. 17 illustrates a close up view of a modified coronal sectional view of a patient's heart with the apparatus shown in FIG. 11 extending therein.

FIG. 18 illustrates a close up view of a modified coronal sectional view of a patient's heart and representation of closure of a patent foramen ovale.

FIG. 19 illustrates a close up view of a modified coronal sectional view of a patient's heart with an apparatus extending therein.

FIG. 20 illustrates a close up view of a modified coronal sectional view of a patient's heart with an apparatus extending therein.

### DETAILED DESCRIPTION

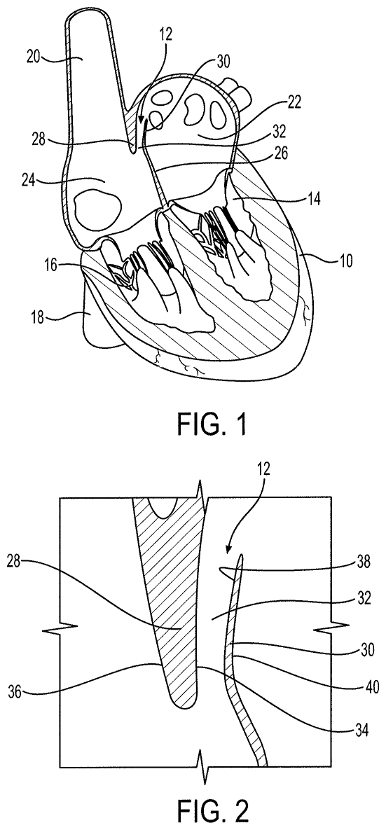

FIG. 1 illustrates a modified coronal sectional view of a patient's heart 10 having a patent foramen ovale (PFO) 12. Features of the patient's heart are visible, including the left ventricle 14, the right ventricle 16, the inferior vena cava 18, the superior vena cava 20, the left atrium 22, and the right atrium 24. The lower portion of the interatrial septum 26 is visible between the left atrium 22 and the right atrium 24. The PFO 12 results from the septum secundum 28 being separated from the septum primum 30, thus causing a tunnel 32 to be positioned between the septum secundum 28 and the septum primum 30.

FIG. 2 illustrates a close up view of the PFO 12 shown in FIG. 1. The septum secundum 28 may include a tunnel surface 34 and an atrial surface 36. The septum primum 30 may similarly include a tunnel surface 38 and an atrial surface 40. The tunnel 32 may be positioned between the respective tunnel surfaces 34, 38.

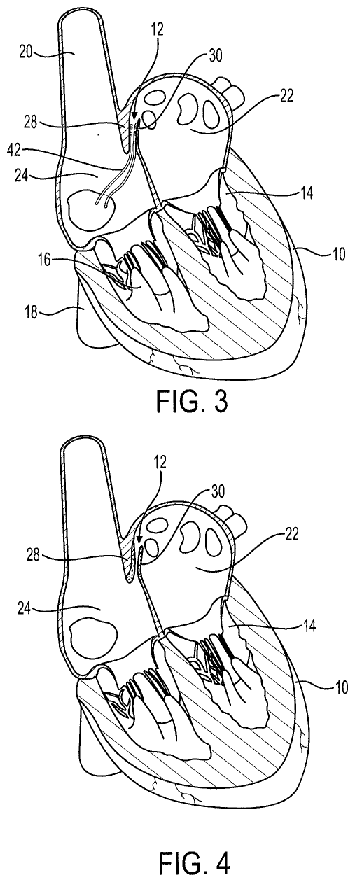

A method for treating the PFO 12 may include ablation of the respective tunnel surfaces 34, 38 of the septum secundum 28 and the septum primum 30. Referring to FIG. 3, an ablation device 42 may be utilized to cause ablation selectively of the tunnel surfaces 34, 38 of the septum secundum 28 and the septum primum 30, which may result in injury, inflammation, swelling, and endothelial denudation. The ablation device 42 as shown in FIG. 3 may include electrodes forming a portion of an electrode catheter, and may apply radio-frequency (RF) energy (either unipolar or bipolar) to the respective tunnel surfaces 34, 38 of the septum secundum 28 and the septum primum 30 to cause the ablation. In other embodiments, other forms of ablation devices 42 may be utilized. For example, ablation devices may be utilized to apply ultrasound, laser, microwave, cryothermal, or pulsed electric fields to cause the ablation. In one embodiment, the ablation device may provide heated fluid that may be circulated to cause the ablation. In one embodiment, the ablation may be provided by an ablation device in the form of one or more inflatable balloons having electrodes coupled thereto, either on the surface of the one or more inflatable balloons or within the one or more inflatable balloons. The one or more inflatable balloons may be positioned within the tunnel 32 of the PFO 12 to cause ablation of the tunnel surfaces 34, 38.

The ablation device 42 may be inserted through a catheter, and may be inserted transvenously to access the PFO 12. Such an entry may allow for a minimally invasive access to the PFO 12. For example, FIG. 3 illustrates the ablation device 42 accessing the PFO 12 via the inferior vena cava 18, although in other embodiments other access methods (e.g., via the superior vena cava 20 or other access points) may be utilized. The ablation device 42 may be on a balloon that may be steerable. In other embodiments, more invasive methods may be utilized to access the PFO 12 including a thoracotomy or other invasive methods.

Upon the ablation device 42 being positioned as desired between the tunnel surfaces 34, 38 of the septum secundum 28 and the septum primum 30, the tunnel surfaces 34, 38 of the septum secundum 28 and the septum primum 30 may be ablated. The ablation may result in injury, inflammation, swelling, and endothelial denudation along opposing tunnel surfaces 34, 38 of the septum secundum 28 and the septum primum 30. FIG. 4, for example, illustrates the tunnel surfaces 34, 38 of the septum secundum 28 and the septum primum 30 following ablation, in which injury, inflammation, swelling, and endothelial denudation has occurred.

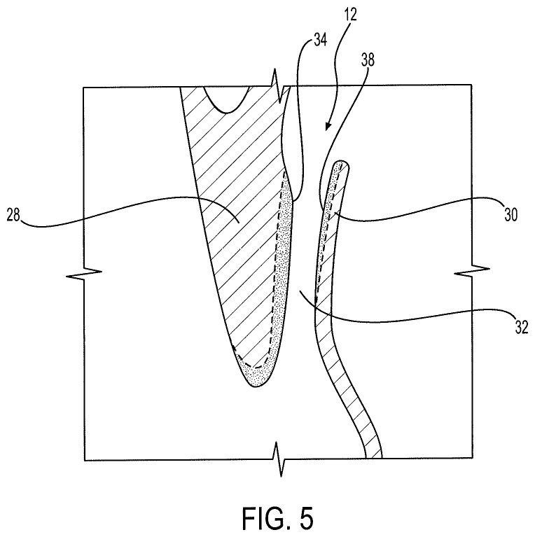

Certain patients may have different sizes or widths (e.g., diameter) of the tunnel 32 of the PFO 12 than others. In certain patients, the tunnel 32 size may be such that injury and endothelial denudation of the septum secundum 28 and the septum primum 30 in the manner shown in FIG. 3 may be sufficient to cause the tunnel surfaces 34, 38 of the septum secundum 28 and the septum primum 30 to spontaneously come into contact with each other, develop adhesions and fuse together, thus closing and treating the PFO 12. As such, following the process shown in FIGS. 3 and 4, the tunnel surfaces 34, 38 of the septum secundum 28 and the septum primum 30 may be in contact with each other, thus treating and closing the PFO 12. In certain other patients, however, the tunnel 32 size may be sufficiently large that further measures may be needed to cause approximation of the tunnel surfaces 34, 38 of the septum secundum 28 and the septum primum 30. A relatively large tunnel 32 size may be likely to be the case with a PFO 12 with an aneurysmal fossa ovalis, or a generally large PFO. Such a PFO 12 may be considered to have a high risk for a paradoxical embolus, other forms of strokes, and may be considered to be a high risk for other maladies of the patient generally. FIG. 5, for example, illustrates a close up view of the PFO 12 shown in FIG. 4, in which ablation has resulted in some swelling of the tunnel surfaces 34, 38 of the septum secundum 28 and the septum primum 30, yet insufficient to close the tunnel 32. The possibility of maladies for the patient therefore remains.

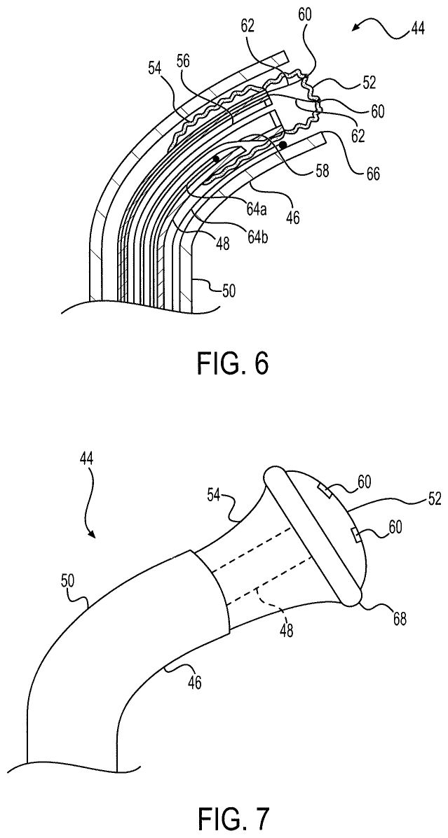

FIG. 6 illustrates a cross sectional view of an apparatus 44 that may be utilized to treat a PFO 12 of a patient. The apparatus 44 may include a catheter 46 having an inner shaft 48 and an outer shaft 50. The apparatus 44 may include a pusher balloon 52 and a support balloon 54, and lumina 56, 58 for filling the respective pusher balloon 52 and support balloon 54. The apparatus 44 may include pressure sensors 60 and electrical conduits 62 to provide signals from the pressure sensors 60. The apparatus 44 may include steering devices 64 to allow for steering of the catheter 46 and accordingly the apparatus 44.

The catheter 46 may comprise an elongated shaft that may include multiple components. The catheter may be configured to extend within at least a portion of a patient's heart and may deliver the pusher balloon 52 to a desired location within the patient's heart. The catheter 46 may be configured to be steerable, and may include the steering devices 64 that allow for steering of the catheter 46 and accordingly the apparatus 44. The steering devices 64 may include wires, such as pull wires, that allow the catheter 46 to steer. A steering device 64a as shown in FIG. 6 may comprise a pull wire that is coupled to the inner shaft 48 and allows for steering of the inner shaft 48, and a steering device 64b may comprise a pull wire that is coupled to the outer shaft 50 and allows for steering of the outer shaft 50. The steering devices 64a, b may be operated in combination to allow for steering of the catheter 46 and accordingly the apparatus 44.

The catheter 46 may have a distal end from which the pusher balloon 52 extends. For example, as shown in FIG. 6, a distal end of the outer shaft 50 may have an opening 66 that the inner shaft 48 and the pusher balloon 52 pass through to extend distally from the outer shaft 50. The inner shaft 48 may be configured to slide relative to the outer shaft 50 such that the inner shaft 48 and pusher balloon 52 may extend distally from the outer shaft 50. The inner shaft 48 may accordingly be able to extend distally (in a deployed configuration) and proximally (in a retracted or undeployed configuration) relative to the outer shaft 50 and may be positioned within the outer shaft 50.

The pusher balloon 52 as shown in FIG. 6 is in an undeployed, or unexpanded or uninflated configuration. The pusher balloon 52 in such a configuration may be configured to fit within the lumen of the outer shaft 50 as shown in FIG. 6. The pusher balloon 52 may be coupled to the inner shaft 48 and configured to advance distally relative to the outer shaft 50 by the inner shaft 48 being advanced distally, or otherwise by being inflated. The pusher balloon 52 may be configured to inflate, to move to an expanded or deployed configuration, in which the pusher balloon 52 has a greater diameter than the diameter of the outer shaft 50 and accordingly the diameter of the catheter 46. The pusher balloon 52 in an inflated, expanded, or deployed state is shown in FIG. 7. The pusher balloon 52 as shown in FIG. 7 has a dome-like shape, with a supporting ring 68. The pusher balloon 52 may be configured to not be compliant, and may be configured to apply a pushing force to a surface of the septum secundum 28 to push the septum secundum 28 towards the septum primum 30. The pushing force may be transmitted via the inner shaft 48, and accordingly via the catheter 46.

The support balloon 54, as shown in FIG. 6, is in an undeployed, unexpanded, or uninflated configuration in which the support balloon 54 is coupled to the inner shaft 48 and is within the lumen of the outer shaft 50. The support balloon 54 may be positioned proximal of the pusher balloon 52. The support balloon 54 may be configured to be semi-compliant, or more compliant that the pusher balloon 52 in certain embodiments. Upon the inner shaft 48 advancing distally, the support balloon 54 may be inflated to the deployed or expanded configuration and may support the pusher balloon 52 and the inner shaft 48 as shown in FIG. 7. For example, the support balloon 54 may resist proximal movement of the pusher balloon 52 upon the pusher balloon 52 pressing against the septum secundum 28 and may resist movement (e.g., bending) of the portion of the inner shaft 48 that the support balloon 54 is positioned around (e.g., encircles). The support balloon 54 may have a cone-like shape as shown in FIG. 7, or another shape as desired (e.g., cylindrical).

The lumina 56, 58 as shown in FIG. 6 may comprise conduits that are configured to extend along the length of the catheter 46 and may be configured to fill the respective pusher balloon 52 and support balloon 54. The lumina 56, 58 may be configured to fill the respective pusher balloon 52 and support balloon 54 with fluid to cause the inflation, and may be configured to withdraw fluid from the respective pusher balloon 52 and support balloon 54 to deflate, unexpand, or undeploy the respective pusher balloon 52 and support balloon 54.

The pressure sensors 60 may be positioned at a distal end of the pusher balloon 52 and may be configured to detect a pressure applied by the pusher balloon 52 to a surface of the patient's heart, including the septum secundum 28. The pressure sensors 60 may be configured to transmit an electrical signal to a user (e.g., a physician, surgeon, or other medical professional) via the electrical conduits 62 to indicate an amount of pressure applied by the pusher balloon 52. The user may be able to accordingly determine the amount of pressure applied by the pusher balloon 52 to determine if the treatment of the PFO 12 is proceeding as desired. In other embodiments, other locations of the pressure sensors 60 may be utilized. Pressure sensors 60 may include piezoelectric sensors, capacitive sensors, electromagnetic sensors, strain sensors, and optical sensors, among others. In addition, other forms of sensors may be utilized such as proximity sensors or the like.

FIG. 7 illustrates a side view of the apparatus 44 with the apparatus in a deployed configuration. The pusher balloon 52 and support balloon 54 are both expanded, and configured to provide a force to the septum secundum 28. Although a single pusher balloon 52 and support balloon 54 are shown in FIGS. 6 and 7, in other embodiments a different number of balloons may be utilized (e.g., one or more pusher balloon 52 and support balloon 54) or a combined pusher balloon 52 and support balloon 54 may be utilized as desired. For example, a balloon having different levels of compliance in different portions of the balloon may be provided.

In operation, the apparatus 44 may be directed to a desired location in the patient's heart in an undeployed or unexpanded configuration. The pusher balloon 52 and support balloon 54 may be in an undeployed, unexpanded, or inflated configuration. The ablation of the tunnel surfaces 34, 38 of the septum secundum 28 and the septum primum 30 may have occurred previously, as shown in FIG. 3, or via another method. The apparatus 44 may be utilized to push the septum secundum 28 towards the septum primum 30 to allow the ablated, or raw, tunnel surfaces 34, 38 to contact each other or be otherwise pushed towards each other. The apparatus 44 may be utilized to fuse the ablated tunnel surfaces 34, 38 in a situation where mere ablation alone has not resulted in spontaneous fusion, such as a situation involving a relatively large tunnel 32 size discussed above.

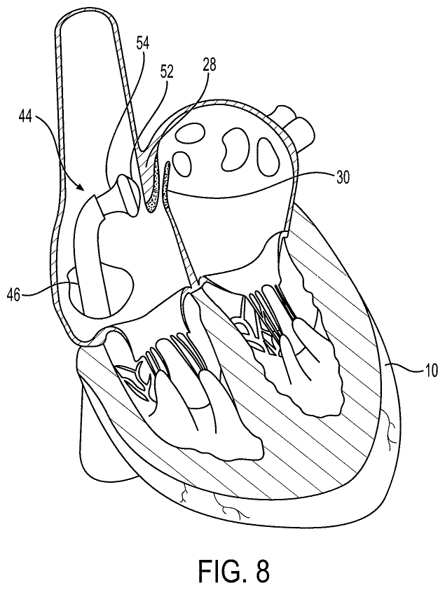

As shown in FIG. 8, the pusher balloon 52 and support balloon 54 may be inflated, deployed, and expanded, and then the pusher balloon 52 may be pressed against the atrial surface 36 of the septum secundum 28 towards the septum primum 30. The pressure sensors 60 may be utilized to determine the presence or amount of contact force applied by the pusher balloon 52. The contact force may cause the tunnel surfaces 34, 38 of the septum secundum 28 and the septum primum 30 after ablation-induced injury to contact each other, resulting in fusion, and closing and treating the PFO 12. The pusher balloon 52 and support balloon 54 may then be deflated, undeployed, and unexpanded, and then withdrawn from the patient's heart 10.

The features of the apparatus 44 described may extend to a single one of the components, or each of the described components may be understood to comprise one or more of the components. For example, the pusher balloon 52 may comprise one or more pusher balloons, the support balloon 54 may comprise one or more support balloons, the pressure sensors 60 may comprise one or more pressure sensors, and other components of the apparatus 44 may comprise one or more of such components.

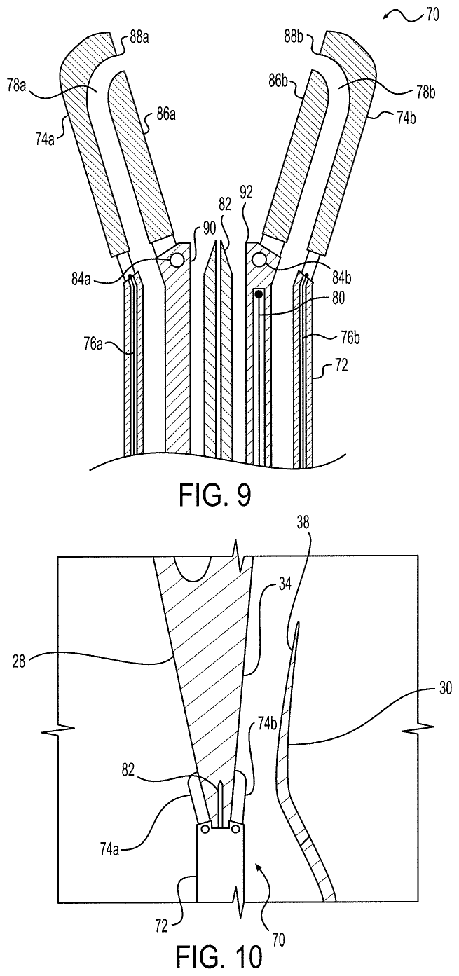

FIG. 9 illustrates a cross sectional view of an apparatus 70 that may be utilized to treat a PFO 12 of a patient. The apparatus 70 may comprise a stabilizing apparatus for stabilizing a portion of a patient's heart in position when material is injected into the portion. The apparatus 70 may be configured stabilize the septum secundum 28 by grasping and holding the septum secundum 28 immobilized and fixed in position while it is being injected with material configured to cause it to swell. The swollen septum secundum 28 is now more likely to come into contact with the septum primum 30 and result in adhesions by swelling in a direction towards the septum primum 30.

The distal end of the apparatus 70 is shown in FIG. 9. The apparatus may include a catheter 72, graspers 74, grasper controls 76, suction devices 78, a steering device 80, and an injection device 82.

The catheter 72 may comprise an elongated shaft that may include multiple components. The catheter 72 may be configured to extend within at least a portion of a patient's heart and may deliver the graspers 74 to a desired location within the patient's heart. The catheter 72 may be configured to be steerable and may include a steering device 80 that allows for steering of the catheter 72 and accordingly the apparatus 70. The steering device 80 may include wires, such as pull wires, that allow the catheter 72 to steer. A steering device 80 as shown in FIG. 9 may comprise a pull wire that is coupled to a portion of the catheter 72 and allows for steering of the catheter and accordingly the apparatus 70.

The catheter 72 may have a distal end from which the graspers 74 extend. The graspers 74 may be configured to grasp a portion of the patient's heart, which may comprise the septum secundum 28. The graspers 74 may comprise arms, or another form of grasper as desired. The graspers 74 as shown in FIG. 9 are pivotally coupled to the catheter 72 with pivot devices 84. The graspers 74a, 74b accordingly may be configured to move (e.g., pivot) towards each other to grasp, and may move (e.g., pivot) to release a portion of the patient's heart. The graspers 74a, 74b may have smooth interior surfaces 86a, b facing each other, or in other embodiments a grip structure may be provided, such as serrations or another grip structure. The grasper controls 76a, b may be utilized to control respective graspers 74a, 74b and move the graspers 74a, 74b to grasp the portion of the patient's heart. The grasper controls 76a, b may comprise wires extending along the catheter 72, or may have another form in other embodiments.

The suction devices 78 may be utilized to apply suction to the grasped portion of the patient's heart, to further stabilize the grasped portion in position. The suction devices 78a, b as shown in FIG. 9 may comprise suction tubes, with respective openings 88a, 88b that are applied to the portion of the patient's heart. The openings 88a, 88b may be positioned on the interior surfaces 86a, b of the graspers 74a, b. The lumina of the suction tubes may extend along the catheter 72 to allow the suction force to be transmitted.

The injection device 82 may be configured to inject material into the portion of the patient's heart that is grasped. The injection device 82 as shown in FIG. 9 may comprise a needle, although in other embodiments other forms of injection devices 82 may be utilized. The needle may include an internal lumen for passing the material into the punctured portion of the patient's heart. The injection device 82 as shown in FIG. 9 may extend along an interior lumen 90 of the catheter 72 and may pass through an opening 92 at a distal end of the catheter 72. The opening 92 may be positioned between the graspers 74a, b or may be provided in another position as desired.

In operation, the apparatus 70 may be directed to a desired location in the patient's heart. The graspers 74 may be in an unexpanded or undeployed configuration. The ablation of the tunnel surfaces 34, 38 of the septum secundum 28 and the septum primum 30 may have occurred previously, as shown in FIG. 3, or via another method. The apparatus 44 may be positioned adjacent the septum secundum 28, and particularly the caudal limit of the septum secundum 28.

Referring to FIG. 10, the apparatus 70 is shown in relation to the septum secundum 28. The graspers 74 may then be actuated to grasp the septum secundum 28. The apparatus 70 may stabilize the septum secundum 28 of the patient's heart by applying the graspers 74 to the septum secundum 28. The apparatus 70 may stabilize the septum secundum 28 in position when the injection device 82 punctures the septum secundum 28 and injects material into the septum secundum 28. The stabilization of the septum secundum 28 via the graspers 74, and also the suction devices 78, may improve the precision of the deposition of material within the septum secundum 28. Upon the septum secundum 28 being stabilized, the injection device 82 may penetrate the septum secundum 28 to a desired degree, and may deposit material within the septum secundum 28 at a desired location. The apparatus 70 may inject the septum secundum 28 with material to cause swelling of the injected septum secundum 28 in a direction towards the opposing septum primum 30.

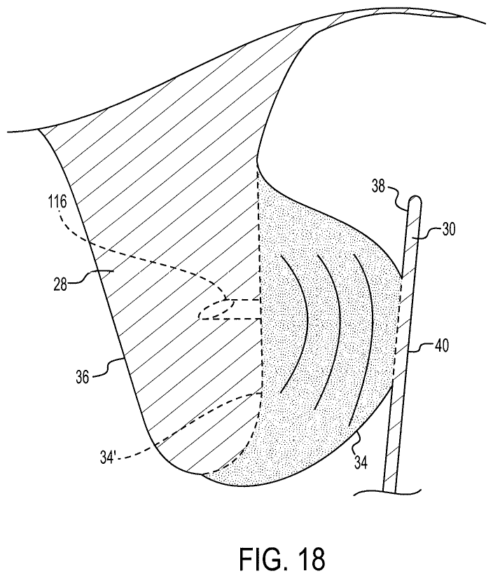

The material injected may comprise material configured to cause swelling of the injected septum secundum 28. The material may comprise one or more of a hydrogel, a hypertonic fluid (e.g., mannitol), an inflammatory fluid (e.g., fluid promoting edema), heparin, or a tissue plasminogen activator (tPA). In other embodiments, other materials may be utilized. The location of the deposition may be such that the swelling is in a direction towards the opposing septum primum 30. The swelling may cause the ablated, or raw, tunnel surfaces 34, 38 to contact each other or otherwise move towards each other. The apparatus 70 may be utilized to fuse the ablated tunnel surfaces 34, 38 in a situation where mere ablation alone has not resulted in spontaneous fusion, such as a situation involving a relatively large tunnel 32 size discussed above. Swelling results in the injected septum secundum 28 bulging in a direction towards the opposing septum primum 30, greatly increasing the chances of fusion is shown in FIG. 18, and such swelling and fusion may result from the use of the apparatus 70. The resulting fusion may close and treat the PFO 12. The apparatus 70 may then be released from the septum secundum 28, and then withdrawn from the patient's heart 10.

The features of the apparatus 70 described may extend to a single one of the components, or each of the described components may be understood to comprise one or more of the components. For example, the graspers 74 may comprise one or more graspers, the steering device 80 may comprise one or more steering devices, the suction devices 78 may comprise one or more suction devices, and other components of the apparatus 70 may comprise one or more of such components. For example, one or more injection devices 82 may be utilized as desired.

In one embodiment, the apparatus 70 may be utilized to stabilize and inject material into the septum primum 30 in a similar manner as described above regarding the septum secundum 28. In one embodiment, the apparatus 70 may be utilized to stabilize and inject material into both the septum primum 30 and the septum secundum 28. The apparatus 70 may stabilize at least one of the septum secundum 28 or the septum primum 30 of the patient's heart. The apparatus 70 may inject at least one of the septum secundum 28 or the septum primum 30 with material to cause swelling of the injected septum secundum 28 or the injected septum primum 30 in a direction towards the opposing septum primum 30 or the opposing septum secundum 28. The apparatus 70 may stabilize the septum secundum 28 or the septum primum 30 of the patient's heart by applying one or more graspers 74 to the septum primum 30 or the septum secundum 28. The injection device 82 may be configured to inject material into at least one of the septum primum 30 or the septum secundum 28 to cause swelling of the injected septum primum 30 or the injected septum secundum 28 in a direction towards an opposing septum primum 30 or an opposing septum secundum 28.

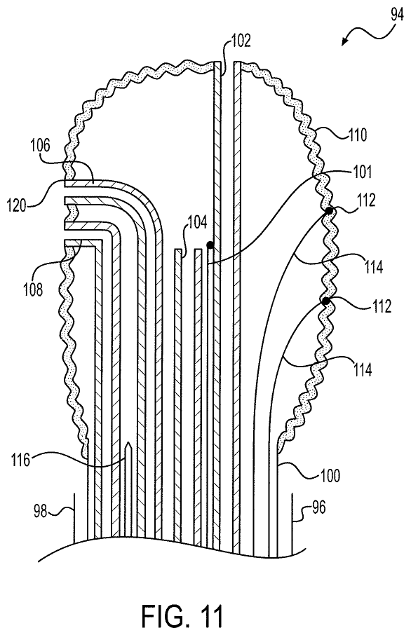

FIG. 11 illustrates a cross sectional view of an apparatus 94 that may be utilized to treat a PFO 12 of a patient and may be configured to be deployed within the PFO tunnel or similar channel within the body. The apparatus 94 may comprise a stabilizing apparatus for stabilizing a portion of a patient's heart by immobilizing and holding the portion of a patient's heart fixed in position when material is injected into the portion. The distal end of the apparatus 94 is shown in FIG. 11.

The apparatus 94 may include a catheter 96 having an outer shaft 98 and an inner shaft 100. The apparatus may include a steering device 101 to allow for steering of the catheter 96 and accordingly the apparatus 94. The inner shaft 100 may include one or more lumina. The lumina may include a guide wire lumen 102, an inflation lumen 104, an injection device lumen 106, and a suction lumen 108. The apparatus 94 may include a stabilizer balloon 110. The apparatus 94 may include ablation devices 112, which may include electrical conduits 114. The apparatus 94 may include an injection device 116.

The catheter 96 may comprise an elongated shaft that may include multiple components. The catheter 96 may be configured to extend within at least a portion of a patient's heart and may deliver the stabilizer balloon 110 to a desired location within the patient's heart. The catheter 96 may be configured to be steerable and may include the steering device 101 that allows for steering of the catheter 96 and accordingly the apparatus 94. The steering device 101 may include a wire, such as a pull wire, that allows the catheter 96 to steer. A steering device 101 as shown in FIG. 11 may comprise a pull wire that is coupled to the guide wire lumen 102 and allows for steering of the catheter 96. In other embodiments other forms of steering devices (e.g., one or more pull wires or other forms of steering devices) may be utilized, including one or more pull wires coupled to other portions of the catheter 96.

The catheter 96 may have a distal end, or distal portion, at which the stabilizer balloon 110 is positioned. The stabilizer balloon 110 may be coupled to the inner shaft 100 at the proximal end or distal portion of the catheter 96. The inner shaft 100 may be configured to slide relative to the outer shaft 98 such that the inner shaft 100 and stabilizer balloon 110 may extend distally from the outer shaft 98. The inner shaft 100 may accordingly be able to extend distally (in a deployed configuration) and proximally (in a retracted or undeployed configuration) relative to the outer shaft 98 and may be positioned within the outer shaft 98. The inner shaft 100 may extend distally from the outer shaft 98 to expose the stabilizer balloon 110 when desired.

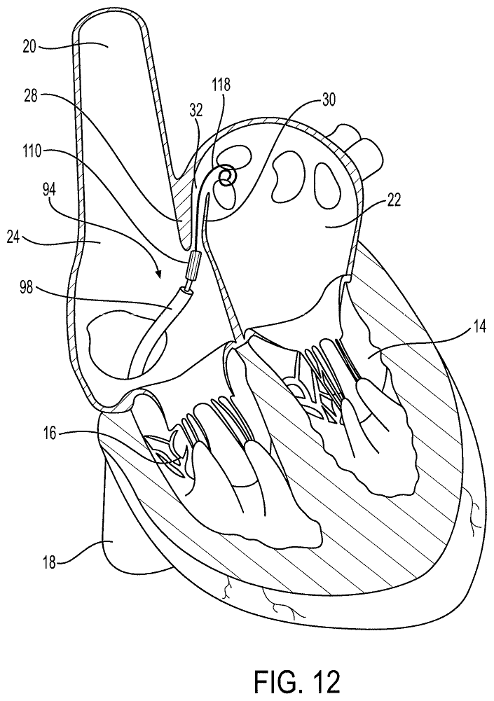

The lumina of the inner shaft 100 may include the guide wire lumen 102. The guide wire lumen 102 may extend along the length of the catheter 96 to allow the catheter 96 to slide along a path created by a guide wire 118 (marked in FIG. 12). The lumina of the inner shaft 100 may include the inflation lumen 104. The inflation lumen 104 may comprise a conduit to allow for inflation or deflation of the stabilizer balloon 110. The inflation lumen 104 accordingly may have a distal end with an opening that opens into an interior of the stabilizer balloon 110, for transmission of liquid or another inflation material into the stabilizer balloon 110.

The lumina of the inner shaft 100 may include the injection device lumen 106, which may allow the injection device 116 to pass through the lumen 106 to puncture a surface of the patient's heart to inject material therein. The injection device lumen 106 may have a distal end that terminates at an opening 120 in the stabilizer balloon 110. The opening 120 may be an opening of the stabilizer balloon 110 configured for the injection device 116 to pass through to inject material into at least one of the septum primum 30 or the septum secundum 28. The injection device 116 may include a needle (such as shown in FIG. 9), although in other embodiments other forms of injection devices 116 may be utilized. The injection device 116 may be configured to extend through the catheter 96 and through the opening 120 to inject material into at least one of the septum primum 30 or the septum secundum 28 to cause swelling of the injected septum primum 30 or the injected septum secundum 28 in a direction towards an opposing septum primum 30 or an opposing septum secundum 28. In one embodiment, the injection device 116 may include an ablation device, to allow the ablation device to perform injury or cause swelling of a portion of the patient's heart upon being inserted into the portion of the patient's heart. The ablation device may include electrodes or other forms of ablation devices as desired. The ablation device may be positioned on a needle of the injection device 116.

The lumina of the inner shaft 100 may include the suction lumen 108, which may comprise part of a suction device configured to apply suction to an adjacent portion of the patient's heart, to further stabilize the adjacent portion in position, particularly during puncture by the injection device 116. Although shown as separate lumina, in other embodiments, the lumina 102, 104, 106, and 108 may be combined into one or more lumina as desired.

The stabilizer balloon 110 as shown in FIG. 11 is in an undeployed, or unexpanded or uninflated configuration. The stabilizer balloon 110 in such a configuration may be configured to fit within the lumen of the outer shaft 98. The stabilizer balloon 110 may be coupled to the inner shaft 100 and configured to advance distally relative to the outer shaft 98 by the inner shaft 100 being advanced distally. The stabilizer balloon 110 may be configured to inflate, to move to an expanded or deployed configuration, in which the stabilizer balloon 110 has a greater diameter than the diameter of the outer shaft 98 and accordingly the diameter of the catheter 96.

The stabilizer balloon 110 may be configured to inflate, expand, or deploy to stabilize one or more adjacent structures of the heart by immobilizing and holding fixed the one or more adjacent structures of the heart. The stabilizer balloon 110 may be positioned within the tunnel 32 of the PFO 12, and then inflated to apply a force to the septum secundum 28 and the septum primum 30. The force applied to the septum secundum 28 and the septum primum 30 may stabilize one or more of the septum secundum 28 or the septum primum 30 such that the surfaces are immobilized for puncture by the injection device 116. The stabilizer balloon 110 may be coupled to the catheter 96 and may comprise one or more stabilizer balloons configured to extend outward from the catheter 96. The one or more stabilizer balloons may be configured to apply a force to both the septum secundum 28 and the septum primum 30 to fix the septum secundum 28 and the septum primum 30 in position when the injection device 116 injects material into at least one of the septum secundum 28 or the septum primum 30.

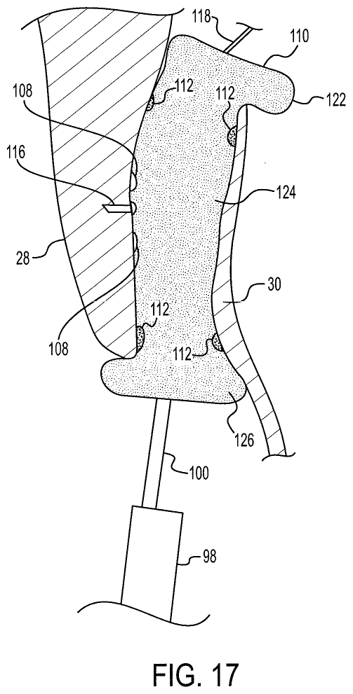

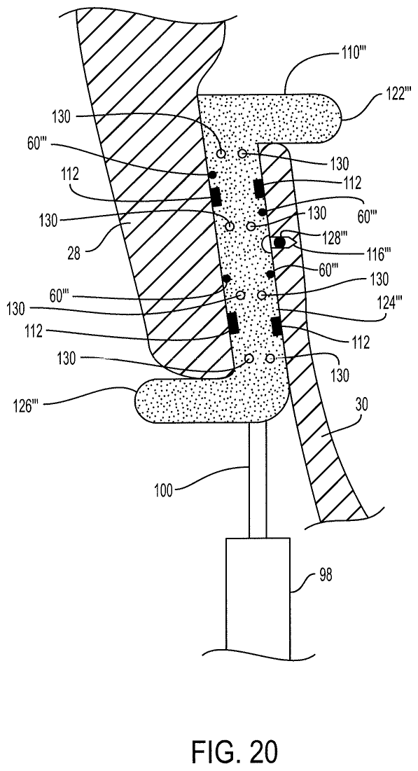

The stabilizer balloon 110 may have a cylindrical shape, or other shape as desired. In one embodiment, as shown in FIG. 17, the stabilizer balloon 110 may have a distal portion 122, a proximal portion 126, and a central portion 124. The distal portion 122 and proximal portion 126 may each have a larger diameter than the diameter of the central portion 124 as shown in FIG. 17. The distal portion 122 may be a cranial portion and the proximal portion 126 may be a caudal portion. The stabilizer balloon 110 may have a dumbbell or “I” shape as shown. The distal portion 122 may be configured to be distal and outside of the tunnel 32 of the PFO 12 and the proximal portion 126 may be configured to be proximal and outside of the tunnel 32 of the PFO 12. The central portion 124 may be configured to be positioned within the tunnel 32 of the PFO 12. Such a shape may serve to further secure the stabilizer balloon 110 within the tunnel 32 of the PFO 12, and stabilize the septum secundum 28 or the septum primum 30, because the overlap and bulge of the distal portion 122 and proximal portion 126 over the ends of the septum secundum 28 or the septum primum 30 may reduce the possibility of axial movement of the stabilizer balloon 110 when in position. The stabilizer balloon 110 may be stabilized in position during an ablation process as well, so that the stabilizer balloon 110 does not dislodge. In one embodiment, the shape of the tunnel 32 of the PFO 12 may be determined, and then the stabilizer balloon 110 may be shaped according to that shape to reduce the possibility of movement of the stabilizer balloon 110 in position. In one embodiment, as shown in FIG. 20, the stabilizer balloon may have a reversed “Z” shape so that the distal and cranial expanded portion bulges into the left atrium and the proximal expanded portion bulges into the right atrium.

The stabilizer balloon 110 may be constructed to be compliant, semi-compliant, or non-compliant as desired. In one embodiment, a central portion 124 may be configured to have less compliance than the distal portion 122 and the proximal portion 126. The central portion 124 may be less compliant to allow the central portion 124 of the stabilizer balloon 110 to deform the PFO 12 upon inflation.

The stabilizer balloon 110 may be configured to perform ablation of adjacent surfaces of the patient's heart. Referring to FIG. 11, the stabilizer balloon 110 may include the ablation devices 112, which may include electrical conduits 114. The ablation devices 112 may be coupled to the stabilizer balloon 110 and may be positioned on or within the stabilizer balloon 110 and configured to ablate a surface of at least one of the septum primum 30 or the septum secundum 28 of the patient's heart. The ablation devices 112 may be configured similarly as shown in FIG. 3, and may include electrodes configured to apply energy in the form of radio-frequency (RF) energy (either unipolar or bipolar) to the respective tunnel surfaces 34, 38 of the septum secundum 28 and the septum primum 30 to cause the ablation. In other embodiments, other forms of ablation devices 112 may be utilized. For example, ablation devices may be utilized to apply ultrasound, laser, microwave, cryothermal, or pulsed electric fields to cause the ablation. In one embodiment, the ablation device may provide heated fluid that may be circulated to cause the ablation. The electrical conduits 114 may couple to the ablation devices 112 for control of the ablation devices 112. In one embodiment, as shown in FIG. 20, the apparatus 94 and particularly the stabilizer balloon 110 may be configured to emit a fluid to allow for greater energy delivery with ablation. The stabilizer balloon 110 may include one or more pores 130 for emitting the fluid from the surface of the balloon 110. The fluid may comprise a saline fluid or other electrically conductive fluid as desired.

In operation, the apparatus 94 may be directed to a desired location in the patient's heart in an undeployed or unexpanded configuration. The stabilizer balloon 110 may be in an undeployed, unexpanded, or uninflated configuration. The ablation of the tunnel surfaces 34, 38 of the septum secundum 28 and the septum primum 30 may have occurred previously, as shown in FIG. 3, or via another method. For example, the stabilizer balloon 110 may include ablation devices 112 that may cause the ablation without a separate device (as shown in FIG. 3) being utilized.

Referring to FIG. 12, the apparatus 94 may be directed to the PFO tunnel 32 in a transcatheter method, which may be transvenous. The apparatus 94 as shown in FIG. 12, is passing into the right atrium 24 via the inferior vena cava 18, although other access methods may be utilized (e.g., via the superior vena cava 20 or other access points). In other embodiments, more invasive methods may be utilized to access the PFO 12 including a thoracotomy or other invasive methods. The apparatus 94 may be steerable via the steering device 101. The apparatus 94 may travel along the guide wire 118 that may have been previously passed into the patient's heart 10 and through the tunnel 32. The apparatus 94 may pass over the guide wire 118 with the guide wire 118 passing through the guide wire lumen 102.

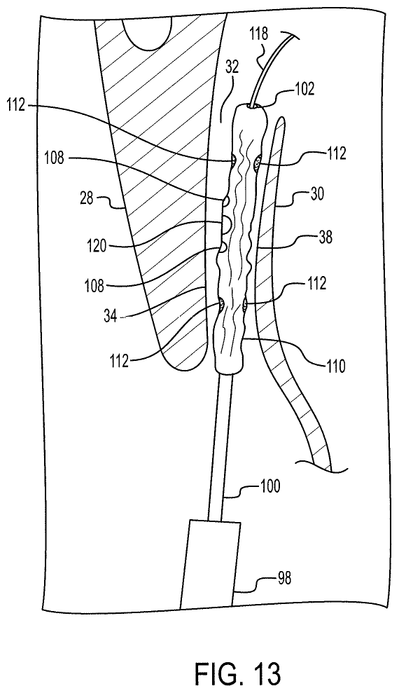

Upon the apparatus 94 approaching the PFO tunnel 32, the stabilizer balloon 110 may be advanced distally out of the outer shaft 98 as shown in FIG. 12. Referring to FIG. 13, the stabilizer balloon 110 in an uninflated, or unexpanded, or undeployed configuration may be passed distally such that the stabilizer balloon 110 is positioned between the tunnel surfaces 34, 38 of the septum secundum 28 and the septum primum 30. The ablation devices 112, the openings of the suction lumina 108, and the opening 120 of the stabilizer balloon 110 that allows the injection device 116 to pass through, may also be positioned between the tunnel surfaces 34, 38 of the septum secundum 28 and the septum primum 30.

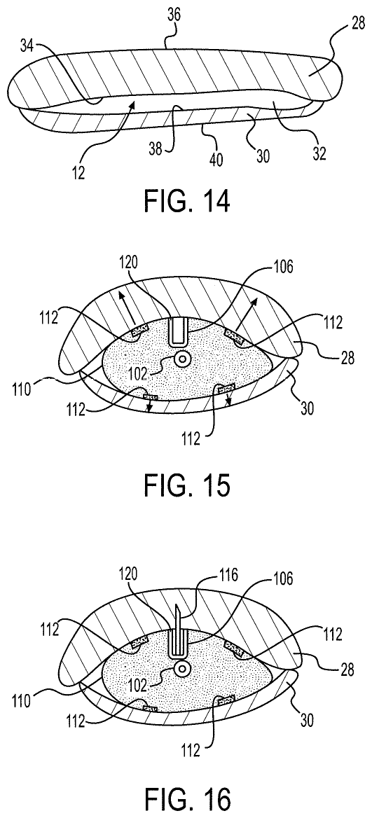

FIG. 14 illustrates a cross sectional top view of the PFO 12 prior to the stabilizer balloon 110 being positioned between the tunnel surfaces 34, 38 of the septum secundum 28 and the septum primum 30. The size of the tunnel 32 is relatively small, with a small diameter relative to the size with the stabilizer balloon 110 positioned therein and inflated.

FIG. 15 illustrates a cross sectional top view of the PFO 12 with the stabilizer balloon 110 being inflated and positioned within the tunnel 32 and between the tunnel surfaces 34, 38 of the septum secundum 28 and the septum primum 30. The inflated stabilizer balloon 110 has exerted a force against at least one of the tunnel surfaces 34, 38 of the septum secundum 28 and the septum primum 30 and has increased the size and diameter of the tunnel 32 in an axis. The axis may be transverse to the length of the tunnel 32 and may be along a transverse plane of the patient's heart or the PFO 12. The apparatus 94 has stabilized at least one of the septum secundum 28 or the septum primum 30. The inflated stabilizer balloon 110 has deformed the tunnel 32, and has stretched the septum secundum 28. In this configuration, the inflated stabilizer balloon 110 is unlikely to slip or otherwise move with respect to the tunnel surfaces 34, 38 of the septum secundum 28 and the septum primum 30. The pressure of the inflated stabilizer balloon 110 stabilizes the septum secundum 28 and the septum primum 30 in position for insertion of the injection device 116. The liquid, or other material filling the stabilizer balloon 110 may be passed through the inflation lumen 104 into the stabilizer balloon 110. The pressure of the inflated stabilizer balloon 110 also stabilizes the stabilizer balloon 110 and the position of the opening 120 of the stabilizer balloon 110, to reduce the possibility of the inflated stabilizer balloon 110 moving during insertion of the injection device 116. The stabilizer balloon 110 may be immobilized and fixed in position.

The ablation devices 112 may be utilized to ablate the tunnel surfaces 34, 38 of the septum secundum 28 and the septum primum 30 when the stabilizer balloon 110 is in position between the septum secundum 28 and the septum primum 30. The ablation devices 112 may cause endothelial denudation, injury, and inflammation to the tunnel surfaces 34, 38 without a separate device needing to be utilized for ablation (e.g., without a device such as the ablation catheter shown in FIG. 3). In other embodiments, a separate device may be utilized for endothelial denudation, injury, or inflammation of the tunnel surfaces 34, 38.

The suction lumina 108 (as marked in FIG. 13) may be utilized to provide suction to further stabilize the septum secundum 28 or the septum primum 30 in position. The suction lumina 108 may be positioned adjacent the opening 120 of the injection device lumen 106 to provide for additional stabilization adjacent the puncture point of the injection device 116.

Referring to FIG. 16, with the septum secundum 28 and the septum primum 30 stabilized in position, the injection device 116 may be passed through the injection device lumen 106 and through the opening 120, and through the tunnel surface 34 of the septum secundum 28. The injection device 116 may penetrate the tunnel surface 34 of the septum secundum 28 to reach a desired portion of the septum secundum 28. If, upon penetration, it appears that the injection device 116 is not in the desired location, then the injection device 116 may be withdrawn, the stabilization reduced, and the stabilizer balloon 110 readjusted to place the injection device lumen 106 and the injection device 116 in the desired location.

The opening 120 of the injection device lumen 106 may be surrounded by the stabilizer balloon 110 such that portions of the stabilizer balloon 110 are positioned distal and proximate the opening 120, and otherwise surround the opening 120 such that the area around the injection point of the injection device 116 is contacted by the stabilizer balloon 110 and stabilized by the stabilizer balloon 110.

Upon the injection device 116 penetrating to the desired location, the septum secundum 28 may be injected with material. FIG. 17, for example, illustrates a side view showing a position of the injection device 116 penetrating the septum secundum 28. The septum secundum 28 may be injected with material to cause swelling of the injected septum secundum 28 in a direction towards the opposing septum primum 30. The material may comprise one or more of a hydrogel, a hypertonic fluid (e.g., mannitol), an inflammatory fluid (e.g., fluid promoting edema), heparin, or a tissue plasminogen activator (tPA) to cause hemorrhage within the structure. In other embodiments, other materials may be utilized. In one embodiment, the materials may include properties that allow for X-ray (which may include fluoroscopy) or other forms of visualization (e.g., echocardiography). The location of the deposition may be such that the swelling is in a direction towards the opposing septum primum 30. In one configuration, the injection device 116 may also be used to deliver ablation energy deep within the septum secundum 28, in an embodiment in which the injection device 116 includes an ablation device (e.g., electrodes on a surface of the injection device 116).

Upon the desired amount and location of injection occurring, the injection device 116 may be withdrawn, and the stabilizer balloon 110 may be deflated, undeployed, and unexpanded to reduce the force applied to the septum secundum 28 and the septum primum 30. The stabilizer balloon 110 may then be withdrawn proximally from the tunnel 32 to allow the tunnel surfaces 34, 38 of the septum secundum 28 and septum primum 30 to contact each other. The apparatus 94 may then be withdrawn from the patient's heart 10.

FIG. 18 illustrates a side illustration of the ablated, or raw, surface 34 of the swollen injected septum secundum 28 contacting the ablated, or raw, surface 38 of the opposing septum primum 30. The swelling may cause the ablated, or raw, tunnel surfaces 34, 38 to contact each other or otherwise move towards each other, greatly increasing the likelihood of development of adhesions. The dashed image of the injection device 116 and the tunnel surface 34′ illustrates the prior position of the tunnel surface and the relative position of the injection device 116. For tissue bridges and collagen cross linking between the septum primum 30 and the septum secundum 28, the ablated or raw surfaces must come in contact with each other. The contact between the ablated, or raw, surface 34 of the swollen injected septum secundum 28 and the ablated, or raw, surface 38 of the opposing septum primum 40 causes the surfaces to fuse together as shown in FIG. 18, thus closing the tunnel 32 and treating and closing the PFO 12.

The apparatus 94 may be utilized to fuse the ablated tunnel surfaces 34, 38 in a situation where mere ablation alone has not resulted in fusion, such as a situation involving a relatively large tunnel 32 size discussed above. Such a large tunnel may be unlikely to result in spontaneous closure and thus the systems, apparatuses, and methods disclosed herein are utilized to produce closure.

The use of the apparatus 94 may beneficially allow for treatment of the PFO 12 without requiring an implant to be left within the patient's heart to close or otherwise treat the PFO 12. In addition, the use of the apparatus 94 may reduce the number of devices that are inserted into the patient's heart to treat the PFO 12, as the apparatus 94 may perform ablation as well as injection of the material that causes swelling of the septum secundum 28 and closure of the PFO 12. The apparatus 94 may be inserted transcatheter and transvascularly, in a minimally invasive manner.

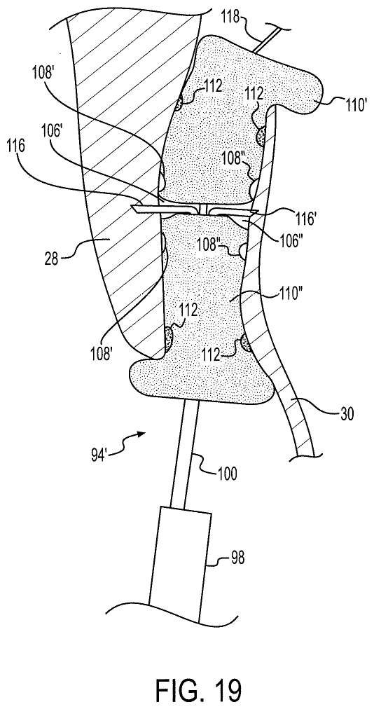

The features of the apparatus 94 described may extend to a single one of the components, or each of the described components may be understood to comprise one or more of the components. For example, the stabilizer balloon 110 may comprise one or more stabilizer balloons, the injection device 116 may comprise one or more injection devices, the ablation devices 112 may comprise one or more ablation devices, and other components of the apparatus 94 may comprise one or more of such components. For example, FIG. 19 illustrates an embodiment of the apparatus 94′ that is similar to the apparatus 94, but includes two of the stabilizer balloons 110′, 110″ providing similar functions as the stabilizer balloon 110. The stabilizer balloons 110′, 110″ may have openings 106′, 106″ positioned between the balloons 110′, 110″ for injection devices 116, 116′ to pass through to puncture the respective septum secundum 28 and the septum primum 30. The injection device 116′ may be configured similarly as the injection device 116, but as shown in FIG. 19 may be configured to puncture and cause swelling of the injected septum primum 30 by injecting the same material described above in regard to swelling of the septum secundum 28 into the septum primum 30. The apparatus 94′ may include suction lumina 108′, 108″ that are configured similarly as the suction lumen 108 and provide additional support for the septum secundum 28 and the septum primum 30. The embodiment shown in FIG. 19 may allow for swelling of both the injected septum secundum 28 and the injected septum primum 30, such that the structures are drawn towards each other (towards the opposing septum primum 30 or the opposing septum secundum 28) to fuse the ablated or raw surfaces in a similar manner as shown in FIG. 18.

FIG. 20 illustrates an embodiment in which a stabilizer balloon 110′″ has a distal portion 122′ and proximal portion 126′ that each have a larger diameter than the diameter of the central portion 124′ as shown in FIG. 20. The distal portion 122′ may be a cranial portion and the proximal portion 126′ may be a caudal portion. The stabilizer balloon 110′″ may have a reversed “Z” shape so that the distal 122′″ and cranial expanded portion bulges into the left atrium with an overlap of the cranial portion of the septum primum 30 and the proximal 126′″ and caudal expanded portion bulges into the right atrium with an overlap of the caudal portion of the septum secundum 28. Such a configuration may assist in stabilizing the stabilizer balloon 110′ within the tunnel 32. Also shown in the embodiment of FIG. 20, are one or more pores 130 for emitting a fluid from the surface of the stabilizer balloon 110′″. The fluid may comprise a saline fluid or other electrically conductive fluid as desired. The fluid may be configured to better allow the stabilizer balloon 110′″ to perform ablation and transmit energy to the septum primum 30 and the septum secundum 28. An ablation device 128′″ on an injection device 116′″ (e.g, a needle) is also shown in FIG. 20 and may be configured similarly as the ablation devices discussed herein.

Also shown in the embodiment of FIG. 20, the stabilizer balloon 110′″ may include pressure sensors 60′″ that are configured to transmit an electrical signal to a user (e.g., a physician, surgeon, or other medical professional) via electrical conduits to indicate an amount of pressure applied by the stabilizer balloon 110′″. The user may be able to accordingly determine the amount of pressure applied by the stabilizer balloon 110″′ to determine if a desired amount of pressure to the PFO 12 is being applied. Pressure sensors 60′″ may include piezoelectric sensors, capacitive sensors, electromagnetic sensors, strain sensors, and optical sensors, among others. In addition, other forms of sensors may be utilized such as proximity sensors or the like.

Also shown in the embodiment of FIG. 20, the injection device 116′″ is penetrating and injecting material into the injected septum primum 30. The injected septum primum 30 may thus swell in a direction towards the opposing septum secundum 28. The methods, systems, and apparatuses disclosed herein may be applied to the septum primum 30 in the same manner as disclosed in regard to the septum secundum 28. For example, the methods shown in FIGS. 12-18 may be applied to the septum primum 30.

The methods, systems, and apparatuses shown in FIGS. 19 and 20 may be applied to, substituted with, or combined with the methods, systems, and apparatuses shown in FIGS. 11-18.

The embodiments described herein may be modified, added to, subtracted from, substituted, or combined across different embodiments. Apparatuses may be used in combination across embodiments as desired. For example, the ablation catheter as described in regard to FIG. 3 may be used to perform ablation across multiple embodiments. The pusher balloon and apparatus 44 as described in regard to FIG. 6 may be used to push the septum secundum 28 towards the septum primum 30 in other embodiments. The apparatus 70 shown and described in regard to FIG. 9 may be utilized to stabilize portions of the heart in various embodiments. In other embodiments, the systems, apparatuses, and methods described herein may be used separately.

The apparatuses described herein may not only comprise apparatuses, but also systems of the components described herein. The scope of the disclosure is not limited to the apparatuses and systems disclosed herein, but also the methods of utilizing such apparatuses and systems. The apparatuses, systems, and methods disclosed herein may be utilized for treating not only a patent foramen ovale, but also for treating a channel like structure in the body. The channel like structure may be similar to the patent foramen ovale.

Exemplary embodiments of the disclosure have been disclosed in an illustrative style. Accordingly, the terminology employed throughout should be read in a non-limiting manner. Although minor modifications to the teachings herein will occur to those well versed in the art, it shall be understood that what is intended to be circumscribed within the scope of the patent warranted hereon are all such embodiments that reasonably fall within the scope of the advancement to the art hereby contributed, and that that scope shall not be restricted, except in light of the appended claims and their equivalents.

What is claimed is:

1. A system for treating a patent foramen ovale of a patient, the system comprising:
a catheter configured to extend within at least a portion of the patient's heart;
one or more stabilizer balloons coupled to the catheter and configured to be positioned within a tunnel of the patent foramen ovale and inflated to apply a force to at least one of a septum primum or a septum secundum of the patient's heart; and
an injection device configured to inject material into at least one of the septum primum or the septum secundum to cause swelling of the injected septum primum or the injected septum secundum in a direction towards an opposing septum primum or an opposing septum secundum.

2. The system of claim 1, further comprising an ablation device coupled to the one or more stabilizer balloons and configured to ablate a surface of at least one of the septum primum or the septum secundum of the patient's heart.

3. The system of claim 2, wherein the ablation device includes one or more electrodes configured to apply energy to the septum primum or the septum secundum of the patient's heart.

4. The system of claim 1, wherein the one or more stabilizer balloons are configured to apply the force to both the septum primum and the septum secundum of the patient's heart to fix the septum primum and the septum secundum in position when the injection device injects the material into at least one of the septum primum or the septum secundum.

5. The system of claim 1, wherein the one or more stabilizer balloons have an opening for the injection device to pass through to inject the material into at least one of the septum primum or the septum secundum.

6. The system of claim 5, wherein the injection device is configured to extend through the catheter and through the opening to inject the material into at least one of the septum primum or the septum secundum.

7. The system of claim 1, wherein the injection device includes a needle for penetrating at least one of the septum primum or the septum secundum.

8. The system of claim 7, wherein an ablation device is positioned on the needle.

9. The system of claim 1, wherein the one or more stabilizer balloons include a distal portion, a proximal portion, and a central portion, and the distal portion and the proximal portion each have a larger diameter than the central portion, and the central portion is configured to be positioned within the tunnel of the patent foramen ovale and the distal portion and the proximal portion are each configured to be positioned outside of the tunnel of the patent foramen ovale.

10. The system of claim 1, wherein the material includes one or more of a hydrogel, a hypertonic fluid, an inflammatory fluid, heparin, or a tissue plasminogen activator.

11. An apparatus for treating a patent foramen ovale of a patient, the apparatus comprising:
a catheter configured to extend within at least a portion of the patient's heart;
one or more stabilizer balloons coupled to the catheter and configured to extend outward from the catheter to apply a force to at least one of a septum primum or a septum secundum of the patient's heart, the one or more stabilizer balloons having an opening; and
an injection device configured to extend through the opening to inject material into at least one of the septum primum or the septum secundum to cause swelling of the injected septum primum or the injected septum secundum in a direction towards an opposing septum primum or an opposing septum secundum.

12. The apparatus of claim 11, wherein the one or more stabilizer balloons include a distal portion, a proximal portion, and a central portion, and the distal portion and the proximal portion each have a larger diameter larger than the central portion, and the central portion is configured to be positioned within a tunnel of the patent foramen ovale and the distal portion and the proximal portion are each configured to be positioned outside of the tunnel of the patent foramen ovale.

13. A method for treating a patent foramen ovale of a patient, the method comprising:
stabilizing at least one of a septum primum or a septum secundum of the patient's heart; and
injecting at least one of the septum primum or the septum secundum with material to cause swelling of the injected septum primum or the injected septum secundum in a direction towards an opposing septum primum or an opposing septum secundum.

14. The method of claim 13, further comprising ablating a surface of at least one of the septum primum or the septum secundum.

15. The method of claim 13, wherein the step of stabilizing includes applying one or more graspers to the septum primum or the septum secundum of the patient's heart.

16. The method of claim 13, wherein the step of stabilizing includes:
positioning one or more stabilizer balloons within a tunnel of the patent foramen ovale and inflating the one or more stabilizer balloons to apply a force to at least one of the septum primum or the septum secundum of the patient's heart.

17. The method of claim 16, wherein the step of injecting includes passing an injection device through an opening of the one or more stabilizer balloons to inject the septum primum or the septum secundum with the material.

18. The method of claim 17, wherein the one or more stabilizer balloons include a distal portion, a proximal portion, and a central portion, and the distal portion and the proximal portion each have a larger diameter than the central portion, and the central portion is configured to be positioned within the tunnel of the patent foramen ovale and the distal portion and the proximal portion are each configured to be positioned outside of the tunnel of the patent foramen ovale.

19. The method of claim 16, further comprising deflating the one or more stabilizer balloons and withdrawing the one or more stabilizer balloons from the tunnel to allow the septum primum and the septum secundum to contact each other.

20. The method of claim 13, wherein the material includes one or more of a hydrogel, a hypertonic fluid, an inflammatory fluid, heparin, or a tissue plasminogen activator.
